(19) United States
(12) Patent Application Publication
(10) Pub. No.: US 2023/0363817 A1
(43) Pub. Date: Nov. 16, 2023

(54) Medical Instrument for Minimally Invasive Removal of Biological Material from a Human or Animal Body

(71) Applicants: Frank Schure, Biesenthal (DE); Viola Schure, Biesenthal (DE)

(72) Inventors: Frank Schure, Biesenthal (DE); Viola Schure, Biesenthal (DE)

(21) Appl. No.: 18/027,926

(22) PCT Filed: Sep. 24, 2021
(86) PCT No.: PCT/EP2021/076414

§ 371 Date: Mar. 23, 2023

(30) Foreign Application Priority Data
Sep. 24, 2020 (DE) 10 2020 124 917.9

(51) Int. Cl. A61B 18/14

(52) U.S. Cl. CPC A61B 18/1482; A61B 18/1487; A61B 2018/144; A61B 2018/00601

(57) ABSTRACT

Described is a medical instrument (1) for the minimally invasive removal of biological material from a human or animal body with a hollow needle (2), which, at a distal end (4) delimiting an interior (3) of the hollow needle (2), includes a peripheral edge (5) having a cutting edge, at least in sections, and with a cutting wire (6), which is arranged such that the cutting wire (6) can be moved relative to the hollow needle (2) at least partially outside the interior (3).

The technical solution described is characterized in that, in the interior (3) of the hollow needle (2), a guide element (7), slidable in the longitudinal direction of the hollow needle (2), is provided, at which the cutting wire (6) is fastened at least at one point and that a means of movement (8) for moving the cutting wire (6) is provided, through which a length of a part of the cutting wire (6) located outside the interior (3) can be changed in a targeted manner as a function of the actuation of a control element (11) acting on the means of movement (8).

### CROSS REFERENCE TO RELATED APPLICATIONS

This application is the U.S. national stage of International Application No. PCT/EP2021/076414, filed on 2021 Sep. 24. The international application claims the priority of DE 102020124917.9 filed on 2020 Sep. 24; all applications are incorporated by reference herein in their entirety.

### BACKGROUND

The invention relates to a medical instrument for the minimally invasive removal of biological material from a human or animal body. The medical instrument includes a hollow needle, which at its distal end has a cutting edge, at least in sections, at a peripheral edge, and a cutting wire, which can be moved at least partially outside the interior of the hollow needle, forming a cutting area.

A variety of different medical instruments for endoscopic procedures, in particular biopsy needles or biopsy probes, resp., are known, using which biological material, such as tumors or parts of tissues or organs, can be removed from the human or animal body. In relation to the removal of tumors, in this context, it is known to punch them and to remove only a part for subsequent pathological examination, or to completely remove a tumor by means of a vacuum biopsy. Since the tumor, however, is cut up and sucked off in small fractions during the vacuum biopsy, upon using this biopsy method, it is not possible to exactly reappraise the resection margins of the tumor pathologically. Therefore, with this method, insights into the original shape, orientation and structure of the tumor within the body cannot be gained. Contrary to the two methods mentioned above, so far, it has only been possible to remove a tumor in its entirety and in its original shape from a patient's body using an open biopsy performed with the aid of an imaging technique.

From EP 0 761 170 A1, an apparatus for removing tissue from a human body is known. The apparatus described includes an elongate base body with an opening at the distal end and with a hollow space for receiving cut-out tissue. Furthermore, a cutting element is provided, which, during the procedure, can be moved transverse to the elongate base body in the immediate vicinity of the opening, so that removed tissue parts are conveyed into the hollow space. Following removal of the respective material, the receiving space filled with tissue can be closed with a blunt closure.

Furthermore, U.S. Pat. No. 5,353,804 A describes an apparatus for removing mammary tumors. The apparatus essentially includes a cutting element movable in a longitudinal direction within a cannula, which has a cutting edge at its distal end. Furthermore, a wire is provided in the interior of the cutting element and arranged coaxially thereto, which is guided into a tissue area on the side opposite the cutting element and is fixed here with an anchor element. With the help of the fixation by the anchor element, the aim is to enable a safe biopsy of the tumor that is as accurate as possible.

DE 38 16 477 A1 describes an apparatus for the removal of biological material from a human body with a shaft having a loop-shaped removal device at one end. This removal device includes a non-cutting scraping edge, through which, during a biopsy, the removal of material is undertaken without a cutting operation. The material scraped off in the patient's body is guided into the interior of the removal device and can thus be removed from the body.

In general, it is a great advantage that, due to the ever-improving diagnostic possibilities, above all attributable to high-quality imaging techniques, comparatively small tumors, in particular mammary tumors, can already be detected in many cases. The problem with the known technical solutions in this context is that with the known apparatuses, the removal of such small tumors cannot be undertaken in a satisfactory manner. Above all the removal of the entire tumor, and thus a reappraisal of the resection margins as exact as possible, cannot be realized with the known apparatuses.

Based on the apparatuses known from the prior art and the problems described above, it is therefore desirable to specify an apparatus for the removal of biological material from a human or animal body, using which even comparatively small tumors, for example mammary tumors, which often have a mean diameter of up to 2 cm, can be removed in their entirety, as true to their original position as possible and clearly demarcated from the surrounding, healthy tissue. It should thus be possible to remove the biological material intended for removal in its entirety, i.e., in one piece, if possible, from the patient's body, and at the same time keep the stress on the surrounding, healthy tissue, and thus on the patient, low. In that, tumor or tissue removal with the apparatus to be specified should in particular be possible to be performed such that the resection margins can be exactly reappraised in the department of pathology. In that, the apparatus to be specified should enable such handling, which the operator of other instruments from prior art is familiar with, and have a comparatively simple structural design, wherein preferably already proven design principles should be used. Furthermore, the apparatus for the removal of biological material to be specified should also enable bipolar application using the generators available on the market. Tumor removal aided by imaging techniques, in particular by means of suitable ultrasonic methods and/or magnetic resonance imaging (MRI), should also be possible.

### SUMMARY

Described is a medical instrument (1) for the minimally invasive removal of biological material from a human or animal body with a hollow needle (2), which, at a distal end (4) delimiting an interior (3) of the hollow needle (2), includes a peripheral edge (5) having a cutting edge, at least in sections, and with a cutting wire (6), which is arranged such that the cutting wire (6) can be moved relative to the hollow needle (2) at least partially outside the interior (3).

The technical solution described is characterized in that, in the interior (3) of the hollow needle (2), a guide element (7), slidable in the longitudinal direction of the hollow needle (2), is provided, at which the cutting wire (6) is fastened at least at one point and that a means of movement (8) for moving the cutting wire (6) is provided, through which a length of a part of the cutting wire (6) located outside the interior (3) can be changed in a targeted manner as a function of the actuation of a control element (11) acting on the means of movement (8).

### DETAILED DESCRIPTION

The above-stated object is solved with a medical instrument according to claim 1. Advantageous embodiments of the invention are the subject of the dependent claims and will be explained in more detail in the following description, in part referring to the figures.

The invention relates to a medical instrument for the minimally invasive removal of biological material from a human or animal body. The instrument includes a hollow needle, which, at a distal end delimiting an interior of the hollow needle, includes a peripheral edge, which has a cutting edge, at least in sections, and a cutting wire, which is arranged such that the cutting wire can be moved relative to the hollow needle at least partially outside the interior. According to the invention, the medical instrument has been further developed to the effect that, in the interior of the hollow needle, a guide element, slidable in the longitudinal direction of the hollow needle, is provided, at which the cutting wire is fastened at least at one point, and that a means of movement for moving the cutting wire is provided, through which a length of a part of the cutting wire located outside the interior can be changed in a targeted manner as a function of the actuation of a control element. Thus, it is essential to the invention that the medical instrument has a cutting wire, which can be inserted into the biological material to be removed and/or the tissue surrounding this material, for example, a tumor, and that the length of the part of the cutting wire located outside the hollow needle can be changed, in particular adapted as required. Using the technical solution according to the invention, it is therefore possible to change the cutting area at least partially surrounded by the cutting wire, in which a biological material intended for removal, in particular a part of an organ or a tissue, is located, also during the procedure and to adapt it to the respective situation. It is thus possible, above all with additional use of suitable imaging techniques, such as ultrasonic imaging methods or magnetic resonance imaging (MRI), to remove exactly that biological material intended for removal from a body in a particularly targeted manner, while sparing healthy tissue as far as possible. In that, the cutting wire is arranged in the interior of the hollow needle as well as fastened at the movable guide element. Advantageously, it is thus possible to move the guide element outward in the longitudinal direction of the hollow needle beyond the distal end of the hollow needle, wherein the cutting wire is moved in the same direction.

Furthermore, it is advantageous, if the cutting wire can be additionally moved relative to the guide element and, in the area of that part of the guide element protruding from the hollow needle, a loop or half-loop can be formed by the cutting wire, which at least partially surrounds the cutting area. According to this embodiment of the invention, by moving the cutting wire relative to the guide element, the size of the loop, and thus of the cutting area, can be adapted to the size of the biological material intended for removal.

In principle, it is conceivable to design the medical instrument operable according to the invention such that it can be used either once or several times. Advantageously, using a medical instrument according to the invention, biological material, in particular tumors, with a mean diameter of up to 2 cm can be removed from a human or animal body. Of great advantage here is that, for example, mammary carcinomas in the breast can already be removed and/or pathologically examined at a comparatively early stage. This is important since, due to the ever-improving imaging techniques, even very small tumors are detected and then removed.

Preferably, the removal of biological material by means of the medical instrument according to the invention is undertaken with the aid of an imaging technique, for example, an ultrasonic imaging method or magnetic resonance imaging, under simultaneous local anesthesia. In that, using suitable imaging techniques ensures safe navigation of the instrument according to the invention within a patient's body as well as exact resection of the biological material intended for removal, sparing the surrounding tissue as far as possible. Above all for the removal of breast tumors, the medical instrument operable according to the invention can be used in a particularly advantageous manner.

According to a particular embodiment of the invention, the means of movement for moving the cutting wire in a targeted manner is designed such that the cutting wire can be formed into a cutting noose or cutting loop at least partially enclosing a cutting area outside the interior of the hollow needle, wherein a size of the cutting area can be changed in a targeted manner as a function of the actuation of an actuation element and the activation of a control element caused hereby, which control element establishes an operative connection between actuation element and means of movement. In that, the operator, i.e., a doctor, performing a minimally invasive procedure with the medical instrument according to the invention is enabled to change the cutting area in a targeted manner by actuating the actuation element, in particular also while the hollow needle is already located within the patient's body. In this context, it is conceivable, for example, that the hollow needle, with the guide element arranged in its interior as well as the cutting wire fastened thereon, is first inserted into the patient, and the guide element with the cutting wire is pushed out from the interior of the hollow needle beyond its distal end only in the area of the biological material to be removed. Furthermore, the cutting wire is moved such that a cutting noose or cutting loop is formed in the area of the biological material to be removed. With a cutting loop adjustable in such way, the resection size can be exactly set and, if needed, also changed during the procedure.

Furthermore, it is conceivable in an advantageous manner that, upon moving the medical instrument up to the area of the biological material to be removed already, the loop is at least partially formed outside the interior of the hollow needle by extending the cutting wire, so that a retraction route, an opening or a gap is created, through which the biological material to be removed can be removed from the body later on. In this context, it is advantageous, if, for the removal of the biological material, in particular a tumor, the guide element with the cutting wire fastened thereon pierces the biological material to be removed in a central location with its distal tip, is passed through the same and is stopped about 3 mm behind the biological material to be removed. Preferably, the cutting wire is now extended, preferably forming the cutting loop, which is adapted to the required resection size as needed. With a 360° or 180° rotational movement in opposite directions, an at least approximately spherical piece of the biological material to be removed is then cut out.

According to a special embodiment, it is conceivable to also move the cutting wire in a suitable manner during the cutting process, so that the shape and/or the size of the cutting area, and thus of the cut-out biological material, is changed. Thus, it is conceivable, for example, that not a spherical piece, but a piece with an at least partially oval shape is cut out.

A particularly advantageously performed minimally invasive procedure with the instrument according to the invention provides that, after the cutting process has been completed, the cutting wire is slightly retracted into the interior of the hollow needle, so that the cutting loop located outside the interior of the hollow needle is shortened or reduced, resp., and thus the cutting area becomes smaller. Preferably, in doing so, the size of the cutting area is halved. Furthermore, while the hollow needle is pulled out from the patient's body, it is advantageous, if the cutting wire is not entirely retracted into the interior of the hollow needle, but preferably remains in the shape of a loop in the cut-out biological material, in order to stabilize the cut-out material during the movement through the patient's body and to thus enable comparatively easy removal.

According to a further particular embodiment of the invention, the guide element with the cutting wire fastened thereon is accommodated in the interior of the hollow needle in a sleeve, arranged coaxially to the wall of the hollow needle, and is slidable within the sleeve in the longitudinal direction. Furthermore, it is advantageous, if the guide element has a recess, which receives at least part of the cutting wire. It is conceivable, for example, that the guide element is designed in a cylindrical or tubular shape and has a notch on its outer side, in which at least part of the cutting wire is arranged. If the cutting wire, fastened at the guide element at least at one point, is moved relative to the guide element, then at least one section of the cutting wire performs a movement within the recess in the longitudinal direction of the guide element.

In a further special embodiment, at least one dilator element is arranged on an outer side of a wall of the hollow needle circumferentially surrounding the interior of the hollow needle, which element can be moved, at least in sections, relative to the wall of the hollow needle from a rest position into an engagement position. Preferably, in that, a surface of the dilator element in its rest position is arranged at least approximately parallel to the surface delimiting the outer side of the wall of the hollow needle. Furthermore, it is advantageous, if the dilator element in its rest position is arranged, at least in sections, coaxially to the wall of the hollow needle and has at least two dilator arms movable in different directions into the engagement position. By means of such a dilator element, thus, in particular before or while the hollow needle is moved out of its engagement position in the body, a gap or an opening can be created in the body, so that the hollow needle with the biological material fastened thereto can be moved out of the patient's body through the surrounding healthy tissue in a comparatively easy manner. In a very special further development, the dilator element includes four dilator arms or wings, which can be moved outward, at least in sections, in a targeted manner.

While retracting the hollow needle with the dilator element extended into its engagement position, the tissue surrounding the hollow needle is thus slightly pushed out of the way, and an enlarged passage opening for the biological material moved is hereby provided. Preferably, the dilator element includes a plurality of arms, wherein it is particularly advantageous, if these dilator arms are part of a dilator sleeve, which in the rest position is oriented coaxially to the wall of the hollow needle and, if needed, can be activated by the operator in a targeted manner. Thus, the dilator element ensures that a return path is dilated in the patient's body, in order to realize a movement of the cut-out biological material through the surrounding healthy tissue as gentle as possible. Furthermore, it is conceivable that the passage opening provided for the removal of the biological material is expanded, for example, by a wire loop, in particular the cutting wire arranged at the hollow needle according to the invention, or by a so-called knife incision.

A very special embodiment of the invention provides that the dilator element includes at least one shape-memory material. In this context, a shape-memory material means a material, which, after deformation from a rest shape into a modified shape, for example by a heat input, and subsequent cooling to the initial temperature, returns to its rest shape.

Apart from that, special shapes of the dilator element, in particular of the arms of the dilator element moving outward upon dilating, are conceivable. Thus, the dilator element, in particular its arms, is preferably curved inward at its distal end, i.e., in the direction of the hollow needle and/or the guide element, still more preferred bent over. In this manner, a facilitated approach of the medical instrument to the biological material to be removed is enabled. It is likewise conceivable to at least slightly dilate the dilator element and/or its arms shaped this way upon moving the cutting wire, in order to thus achieve a slight, blunt dilation.

According to a further special embodiment of the invention, it is provided that the dilator element is designed such that the cut-out biological material is grasped by it and fixed during a movement through the surrounding tissue. In this context, it is advantageous, if the dilator element has a plurality of arms, which are bent over at their distal ends and have a gripping area on their side facing the guide element. A dilator element shaped this way can fix the cut-out material like a gripper with gripping jaws.

Furthermore, in an advantageous embodiment of the instrument according to the invention, a gripping element, which can be moved out of the interior beyond the distal end, is provided for fixation of the cut-out biological material. By means of such a gripping element, the cut-out biological material is fixed, in order to be able to remove it from the body as completely as possible and in its original orientation. In this case, it is conceivable that a respective gripping element is designed such that, after the cutting process has been completed, it can be extended from the interior of the hollow needle beyond its distal end in a targeted manner by actuating an operating element. Subsequently, suitable gripping arms of the gripping element are preferably placed around the cut-out biological material, in order to fix it, but without damaging it.

Alternatively, or in addition to that, it is conceivable that a respective gripping element for fixation of the cut-out biological material is not arranged at the distal end of the hollow needle, but, at least temporally, at the distal end of the medical instrument, which in its engagement position is located on the side of the cut-out biological material opposite the hollow needle.

According to a special further development of the invention, the gripping element is designed, at least partially, movable relative to the guide element and connected to the medical instrument via suitable means of movement. Preferably, the gripping element is partially arranged in the interior of the hollow needle between the wall of the hollow needle and the guide element, wherein, in principle, it is conceivable that the gripping element is circumferentially surrounded by a sleeve concentric to the wall of the hollow needle. Once the cut-out biological material has been gripped by the gripping element, in general, two different scenarios are conceivable for the further procedure. On the one hand, it is possible that the biological material in this position, in which it is fixed by the gripping element, is removed from the body through the surrounding healthy tissue. On the other hand, it is conceivable that the cut-out biological material is transferred into a removal space provided in the interior of the hollow needle by means of the gripping element, and therein is moved out of the body together with the remaining elements of the medical instrument. In this case, the guide element with the cutting wire fastened thereon is also designed such that sufficient space remains in the interior of the hollow needle, in order to be able to provide a removal space for the cut-out biological material.

In a further embodiment, the gripping element is at least partially arranged in the interior of the guide element. In this context, it is conceivable that the guide element includes a cylindrical recess in its interior, in which the gripping element is arranged, wherein the gripping element, particularly preferred, can be moved along the longitudinal axis of the guide element. Regardless of the arrangement of the gripping element either at the end of the distal end of the hollow needle's interior or at the guide element, it is furthermore advantageous, if the gripping element comprises at least one shape-memory material. In this case, too, the shape-memory material is characterized in that, following deformation from a rest shape into a modified shape, for example by a heat input, and subsequent cooling to the initial temperature, it returns to its rest shape.

A further embodiment of the invention provides that an operating situation of the medical instrument can be created, in which the cutting wire is supplied with electrical energy and forms a pair of electrodes with the guide element, the gripping element, an electrically conductive element arranged in the interior of the hollow needle, a sleeve arranged in the interior of the hollow needle or the wall of the hollow needle, wherein a current flow is initiated between the electrodes, so that an electric arc forms in this area. In such an operating situation, the cutting wire advantageously forms the active electrode of the pair of electrodes, wherein a current flow is effected between the electrodes, in order to generate an electric arc and thus at least support the cutting process in the biological material. This operating situation represents a so-called bipolar application, in which a current flows between the two electrodes of the medical instrument and thus only in a defined area of the biological material. Contrary to the monopolar technique, here, the current does not flow extensively through the patient's body, and a neutral electrode is not required.

In a particularly advantageous manner, the wall of the hollow needle or a sleeve arranged in the interior of the hollow needle forms the counter-electrode to the cutting wire representing the active electrode.

In a very special embodiment of the invention, it is provided that the medical instrument includes a channel, in which an endoscopic instrument is located, which enables viewing of a tissue area located in the distal direction in front of the medical instrument. Preferably, the channel is designed such that, during a procedure, an endoscope can be moved through it in the longitudinal direction, in particular extended into the patient's body as well as retracted therefrom. Preferably, the channel is arranged in the interior of the hollow needle, more particularly in the interior of the guide element, and includes a distal opening, through which an optical unit and/or camera of the endoscope can be moved, at least partially, into a tissue area located outside the medical instrument. Alternatively, or in addition thereto, it is conceivable that at least one endoscope, using which a surgical area can be visualized to the operator, is an integral component of the medical instrument. Preferably, an endoscope operable in such manner, in particular its optical unit or camera, is, at least in sections, fastened to the hollow needle or the guide element movable in the longitudinal direction of the hollow needle.

Apart from that, it is particularly advantageous, if a medical instrument designed according to the invention is manufactured from non-magnetic materials. This is above all required, when the medical instrument according to the invention is used with the aid of magnetic resonance imaging (MRI) as an imaging technique for the removal of biological material from a human or animal body.

### BRIEF DESCRIPTION OF THE DRAWINGS

In the following, the invention is set forth in more detail without restriction of the general idea of the invention on the basis of embodiments with reference to the figures. These show:

FIG. 1: lateral view of a medical instrument designed according to the invention for the removal of biological material from a human or animal body;

FIG. 2: top view of the distal end of a hollow needle of a medical instrument designed according to the invention; as well as

FIG. 3: section of a schematic cross-sectional view of the hollow needle of a medical instrument designed according to the invention.

### DETAILED DESCRIPTION OF THE PREFERRED EMBODIMENTS

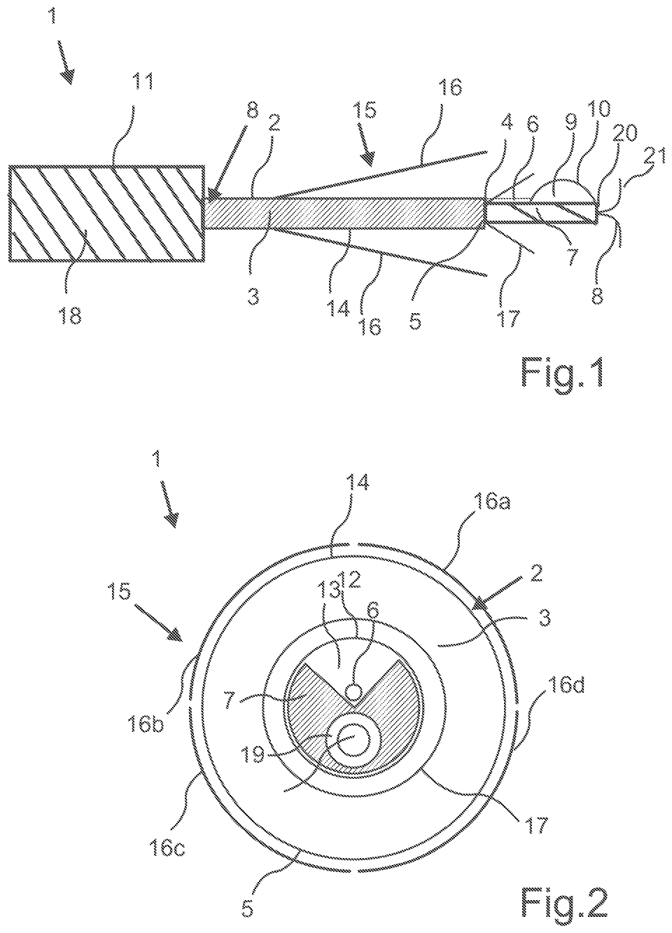

FIG. 1 shows a medical instrument 1 for the removal of biological material, in particular a tumor, such as a mammary carcinoma, from a human or animal body in a lateral view. The instrument 1 includes a handle 18, at which an operator, i.e., in particular a physician performing the procedure, can grab the instrument 1, move it in a suitable manner and initiate different operating functions by actuating at least one control element 11.

Thus, in the area of the handle 18, control elements provided for operating the instrument are arranged, via which the operator can actuate various elements and means of movement 8 of the medical instrument 1, and in particular move a cutting tool, which here is designed as a cutting wire 6 fastened to a likewise movable guide element 7, a gripping element 17 for fixation of the cut-out biological material as well as a dilator element 15.

According to the embodiment shown in FIG. 1, the medical instrument 1 includes a hollow needle 2, designed in the shape of a hollow cylinder or a sleeve, in the interior 3 of which a guide element 7, movably arranged in the longitudinal direction of the hollow needle 2, is provided. In the operating state represented in FIG. 1, the guide element 7 is located in a position, in which the guide element 7 protrudes beyond the distal end 4 of the interior 3 of the hollow needle 2. Furthermore, at the distal end 4 of the guide element 7, a cutting wire 6 is fastened, which can be moved due to manual actuation of a control element 11 and the means of movement 8 activated thereby. Apart from that, the cutting wire 6 is arranged in a recess 13, designed as a notch, which extends in the guide element 7 in its longitudinal direction, and can be moved relative to the guide element 7, so that outside the interior 3 of the hollow needle 2, a cutting loop 10, at least partially enclosing a cutting area 9, can be formed. For that, suitable holding and/or deflection means are provided at the guide element 7, which act on the cutting wire 6 such that, upon moving the cutting wire 6 along the guide element 7 in the direction towards the distal end 20 of the guide element 7, not only the length of the cutting wire 6 located outside the interior 3 is increased, but also a suitable cutting loop 10 is formed. This process is performed by the operator in a controlled manner by actuating at least one control element 11 in the area of the gripping element 17.

According to the embodiment shown in FIG. 1, thus, by moving the cutting wire 6, a cutting loop 10 outside the interior 3 of the hollow needle 2 can be created, which is used as a cutting element. The size of this cutting loop 10, and thus of the cutting area 9 at least partially enclosed by this loop 10, can be adapted by the operator as needed.

Preferably, the removal of biological material, in particular a tumor with a mean diameter of up to 2 cm from the female breast, is undertaken with the aid of an imaging technique, for example an ultrasonic imaging method, a mammography or magnetic resonance imaging (MRI), or imaging techniques that can be used in the future, such as tomosynthesis or rotational computed tomography, so that the operator can reach the tumor in a safe and targeted manner and cut it out of the healthy tissue with high accuracy.

Furthermore, the medical instrument 1 shown in FIG. 1 includes a dilator element 15 on the outer side of the wall of the hollow needle 14 in the form of a sleeve with four dilator arms 16, the distal ends of which can be respectively folded out. As soon as the hollow needle 2 with the cut-out biological material is to be removed from a human or animal body, the operator can actuate the dilator arms 16, again via a control or operating element, so that these respectively move outward with their distal ends and create a wedge shape, so that the cut-out biological material can be moved through a comparatively large passage opening through the surrounding healthy tissue through the body, and finally can be removed therefrom.

Furthermore, a gripping element 17 with four gripping arms is arranged at the distal end 4 of the hollow needle 2, which arms, once the biological material to be removed has been cut out, are placed around the cut-out material, so that it is ensured that the biological material can be removed from the body as completely as possible and in the original orientation it had inside the body. In addition, according to the embodiment shown in FIG. 1, an anchor element 21 is provided at the distal end 20 of the guide element 7, which in a rest position is stored inside the guide element 7, and then, when the hollow needle 2 as well as the guide element 7, as will be explained in more detail further below, are located in the engagement position in the body, is extended from the guide element 7. The anchor element 21 fixes the hollow needle 2 with the guide element 7 in the engagement position during the cutting process.

For the removal of biological material from a human or animal body, in particular a tumor, for example a mammary carcinoma, the hollow needle 2, with the guide element 7 located in the interior 3 of the hollow needle 2 while in the rest position and the cutting wire 6 fastened thereon on one side, is inserted into the breast with the aid of an imaging technique, such as an ultrasonic imaging diagnostic method. In the immediate vicinity of the tumor to be removed, the guide element 7, with the cutting wire 6 fastened thereon, is extended from the interior of the hollow needle 2. It is likewise conceivable to extend the guide element 7 and the cutting wire 6 upon approaching the tumor already, and to thus already create an enlarged passage opening or a gap, resp., by means of the cutting wire 6, in particular a shaped cutting loop 10, through which the hollow needle 2 with the cut-out biological material can be removed from the body later on.

Finally, the guide element 7 with the cutting wire 6 is pierced into the center of the tumor, pushed through it, and the movement is stopped as soon as the distal end 20 of the guide element 7, i.e., the tip of the medical instrument 1, is located about 3 mm behind the tumor. Now the cutting wire 6 is moved such that a cutting loop 10 with exactly the required size is formed, i.e., the required radius is set. In that, the mean diameter of the cut-out biological material is usually about 1 cm larger than that of a tumor to be removed. In this manner, the resection size can be exactly set by shifting the cutting wire 6 and respective formation of a cutting loop 10. With a rotational movement either by 360° or by 180°, respectively in opposite directions, the biological material is finally cut out in the shape of a sphere. It is even possible that the operator moves the cutting wire 6 in the longitudinal direction of the guide element 7, also during the cutting process, if needed, and thus changes the part of the cutting wire 6 located outside the interior 3 of the hollow needle 2, and thus the size of the cutting loop 10. In this case, the cut-out material has a shape deviating from the spherical shape.

Once the biological material intended for removal, here a tumor in the female breast, has been cut out, the cutting wire 6 is retracted into the interior 3 of the hollow needle 2 such that the size of the cutting area 9, and thus of the cutting loop 10, is about halved. This comparatively small cutting loop 10 remains in the tumor to be removed, in order to stabilize it in its longitudinal axis and to guarantee safe and easy removal. Before the tumor is moved through the body, first, a gripping element 17, likewise arranged in the interior 3 of the hollow needle 2, in particular its four-armed gripping claw, is extended and the cut-out tumor fixed by means of the gripping claws. Subsequently, the cut-out tumor is carefully moved out of the body. In order to facilitate this process, in particular to provide a passage opening for the tumor to be removed as large as possible, prior to retraction, the dilator element 15 in the shape of a four-armed, dilating sleeve is actuated, so that the individual dilator arms 16 are moved outward. In this manner, a wedge is created, which generates further facilitation upon removal of the tumor. In order to realize the gentlest possible tumor removal through the surrounding healthy tissue, it is conceivable that the passage opening is already enlarged upon approaching the tumor by means of a cutting loop 10. Preferably, a cutting loop 10 provided and used according to the invention can be supplied with electrical energy and forms a pair of electrodes together with a further electrode, such as the wall of the hollow needle 14 or another element in the interior 3 of the hollow needle 2, between which an electric arc supporting the cutting process is formed. Furthermore, it is conceivable to enlarge the opening for removal of the biological material by means of a knife incision.

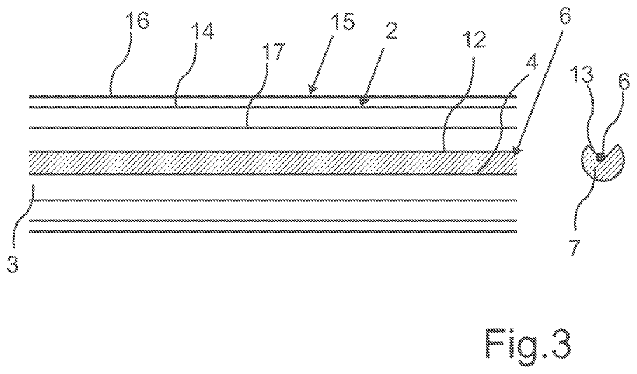

FIG. 2 shows the distal end 4 of a hollow needle 2 of a medical instrument 1 designed according to the invention in a top view. It is clearly visible that the individual components, such as the cutting wire 6, the wall of the hollow needle 14, a guide sleeve 12 for receiving the guide element 7 as well as the guide element 7 with the cutting wire 6 and the anchor element 21 arranged in the interior, are arranged concentrically around a longitudinal center axis of the hollow needle 2.

According to the embodiment shown in FIG. 2, the wall of the hollow needle 14 circumferentially encloses an interior 3, in which a further sleeve, the so-called guide sleeve 12, extends in the longitudinal direction. Within this guide sleeve 12, the guide element 7 is movably arranged in the longitudinal direction. Furthermore, the guide element 7 includes a recess 13 in the shape of a notch extending in the longitudinal direction, in which the cutting wire 6 used as a cutting element is arranged.

Furthermore, the guide element 7 includes a cylindrical borehole 19, likewise extending in the longitudinal direction, in which an anchor element 21 is arranged.

On the outer side of the wall of the hollow cylinder 14, a dilator element 15 is located, which includes four dilator arms 16a, 16b, 16c, 16d arranged in parallel to the outer surface of the wall of the hollow cylinder and shaped in parallel hereto, which can be moved and, upon retraction of the hollow needle 2 from the patient's body, can be distanced from the wall of the hollow needle 14, at least in sections, i.e., can be dilated. In this manner, a wedge can be created as needed, which, upon movement of the hollow needle 2 in the proximal direction, pushes the surrounding, usually healthy tissue out of the way, and thus enlarges the passage opening for passing the hollow needle 2 with the cut-out tumor.

FIG. 3 further shows the hollow needle 2 of a medical instrument 1 designed according to the invention with the remaining components provided in this area in a schematic cross-sectional view. In the interior 3 of the hollow needle 2, the guide element 7 is located, which is arranged in a guide sleeve 12. The guide element 7 includes a recess 13 in the shape of a notch extending in the longitudinal direction for receiving the cutting wire 6, which forms the cutting element.

The cutting wire 6 as well as the guide sleeve 12, within which the guide element 7 is arranged, are connected with a power supply in an electrically conductive manner and, in a special operating situation, for a so-called bipolar application, form a pair of electrodes, of which the cutting wire 6 represents the active electrode. After applying a current of suitable strength, a current flow is initiated between these two electrodes and an electric arc is formed, through which the cutting process can at least be supported. Contrary to known monopolar applications of high-frequency surgery, the bipolar application above all has the advantages that the current flow is locally restricted in the patient's body and no neutral electrode is required.

Furthermore, a gripping element 17 with a plurality of gripping arms, extending in the longitudinal direction of the hollow needle 2, is provided, which arms circumferentially surround the guide sleeve 12 and in that are arranged between the guide sleeve 12 and the wall of the hollow needle 14. At the distal end of this gripping element 17 with the plurality of gripping arms, gripping claws are located, which serve fixation of the cut-out biological material.

On the outer side of the wall of the hollow needle 14 and coaxially hereto, there is a dilator element 15, which includes individual dilator arms 16, which, upon removal of the cut-out biological material from the body, move outward in sections and thus create a passage opening, through which the cut-out biological material can be removed in a comparatively easy manner and in one piece.

### LIST OF REFERENCE NUMERALS

1 Medical instrument
2 Hollow needle
3 Interior of the hollow needle
4 Distal end of the hollow needle
Peripheral edge
6 Cutting wire
7 Guide element
8 Means of movement
9 Cutting area
10 Cutting loop
11 Control element
12 Guide sleeve
13 Recess
14 Wall of the hollow needle
15 Dilator element
16 Dilator arms
17 Gripping element
18 Handle
19 Borehole in the guide element
20 Distal end of the guide element
21 Anchor element

1. A medical instrument (1) for the minimally invasive removal of biological material from a human or animal body with a hollow needle (2), which, at a distal end (4) delimiting an interior (3) of the hollow needle (2), includes a peripheral edge (5) having a cutting edge at least in sections, and with a cutting wire (6), which is arranged such that the cutting wire (6) can be moved relative to the hollow needle (2) at least partially outside the interior (3), characterized in that in the interior (3) of the hollow needle (2), a guide element (7), slidable in the longitudinal direction of the hollow needle (2), is provided, at which the cutting wire (6) is fastened at least at one point, and that a means of movement (8) for moving the cutting wire (6) is provided, through which a length of a part of the cutting wire (6) located outside the interior (3) can be changed in a targeted manner as a function of the actuation of a control element (11) acting on the means of movement (8).

2. The medical instrument (1) according to claim 1,
characterized in that the means of movement (8) is designed such that the cutting wire (6) outside the interior (3) of the hollow needle (2) can be shaped into a cutting loop (10) at least partially enclosing a cutting area (9), wherein a size of the cutting area (9) can be changed in a targeted manner as a function of the actuation of the control element (11).

3. The medical instrument (1) according to claim 1,
characterized in that the guide element (7) with the cutting wire (6) fastened thereon is arranged in a guide sleeve (12) in the interior (3) of the hollow needle (2).

4. The medical instrument (1) according to claim 3,
characterized in that the guide element (7) has a recess (13), at least in sections, in which at least part of the cutting wire (6) is arranged.

5. The medical instrument (1) according to claim 1,
characterized in that, on an outer side of a wall of the hollow needle (14) circumferentially enclosing the interior of the hollow needle (2), at least one dilator element (15) is arranged, which, at least in areas, can be moved relative to the wall of the hollow needle (14) from a rest position into an engagement position.

6. The medical instrument (1) according to claim 5,
characterized in that a surface of the dilator element (15) in the rest position is arranged at least approximately parallel to a surface delimiting the outer side of the wall of the hollow needle (14).

7. The medical instrument (1) according to claim 5,
characterized in that the dilator element (15) in its rest position is arranged sleeve-shaped and coaxially to the wall of the hollow needle (14) and has at least two dilator arms (16a, 16b) movable in different directions into the engagement position.

8. The medical instrument (1) according to claim 5,
characterized in that the dilator element (15) comprises at least one shape-memory material.

9. The medical instrument (1) according to claim 1,
characterized in that a gripping element (17), which can be moved out of the interior (3) beyond the distal end (4), is provided for fixation of the biological material.

10. The medical instrument (1) according to claim 9,
characterized in that the gripping element (17) is arranged in a movable manner relative to the guide element (7).

11. The medical instrument (1) according to claim 9,
characterized in that the gripping element (17) is arranged at least partially in the interior of the guide element (7).

12. The medical instrument (1) according to claim 9,
characterized in that the gripping element is partially arranged in the interior of the hollow needle, between the wall of the hollow needle and the guide element.

13. The medical instrument (1) according to claim 9,
characterized in that the gripping element (17) comprises at least one shape-memory material.

14. The medical instrument (1) according to claim 1,
characterized in that an operating situation can be created, in which the cutting wire (6) is supplied with electrical energy and forms a pair of electrodes with the guide element (7), the gripping element (17), an electrically conductive element arranged in the interior of the hollow needle (2), a sleeve arranged in the interior (3) of the hollow needle (2) or the wall of the hollow needle (14).

15. The medical instrument (1) according to claim 1, exclusively manufactured from non-magnetic materials.
